(19) United States
(12) Patent Application Publication
(10) Pub. No.: US 2009/0148489 A1
(43) Pub. Date: Jun. 11, 2009

(54) BIOABSORBABLE MATERIAL

(76) Inventor: John Joseph Cooper, Cheshire (GB)

Correspondence Address: SMITH-HILL AND BEDELL, P.C., 16100 NW CORNELL ROAD, SUITE 220, BEAVERTON, OR, 97006, US

(21) Appl. No.: 11/719,516

(22) PCT Filed: Nov. 11, 2005
(86) PCT No.: PCT/GB05/04342

§ 371 Date: May 16, 2007

(30) Foreign Application Priority Data
Nov. 24, 2004 (GB) 0425790.3

(51) Int. Cl. B01J 20/26; A61P 19/00; A61K 33/42; A61K 33/06; A61K 33/10; A61K 33/00

(52) U.S. Cl. 424/423; 502/402; 424/602; 424/696; 424/687; 424/724

(57) ABSTRACT

A bioabsorbable material suitable for implanting within a human body, the material including fibers of a composite of a synthetic bioabsorbable polymer such as poly-lactic acid, and a particulate bioactive filler such as calcium phosphate powder. The fibers are discontinuous with non-uniform cross-sections and non-uniform cross-sectional areas. The surface topography provided by the fibers provides a substrate which is more amenable to cellular colonization than prior materials.

This invention concerns bioabsorbable materials suitable for implanting within a human body, and bioabsorbable piece materials suitable for implanting within a human body.

In the fields of surgery and the emerging field of tissue engineering it is desirous to have implantable devices which support and encourage the attachment, differentiation and proliferation of cells and the growth of functional bodily tissue. Tissue engineering is the practice which seeks to repair, regenerate or restore form and function of diseased, damaged or malfunctioning bodily tissue through the application of the principles of engineering and the biological sciences. A temporary framework to support cellular attachment and new tissue growth by providing an appropriate physical and chemical environment is described as a scaffold. The scaffold can be pre-seeded with cells outside the body which are then either culture expanded prior to implantation, mixed with autologous blood, bone marrow or culture expanded autologous cells immediately prior to implantation, or implanted as a sterile material which subsequently becomes infused with the body's fluids and cells which then become part of the healing cascade in the regeneration of new tissue.

To perform as an effective scaffold the material is required to have certain properties and characteristics. It must have a porosity and pore size amenable to cellular infiltration and provide the high permeability necessary to enable ingress of cell nutrients and egress of cellular waste products. The scaffold should have a high internal surface area to maximise the capacity to entrain cells and provide the space for new tissue to grow. The porosity should be fully interconnected with no closed or re-entrant pores. There must be sufficient mechanical integrity to the scaffold to maintain morphological characteristics either in vitro or in vivo until such time as the re-growing tissue can sustain that function. The material of the scaffold should be hydrophilic such that it is easily wetted by bodily fluids and/or cell culture medium and ideally would be at least conducive and preferably inducive to the growth of new tissue. The scaffold should be completely bioabsorbed in a time frame commensurate with its replacement by new tissue. The degradation products of the scaffold material should be non toxic and not impede or inhibit cell proliferation and growth of new tissue.

Many different materials in a wide range of physical forms have been proposed and trialled as bone void fillers and tissue scaffolds. Foamed materials in general, either ceramics or polymers often contain high levels of closed or re-entrant pores and pores with narrowed interconnections. These impede both diffusion and mass transfer and limit the potential for growth of new tissue. Porous ceramics including the bioactive and osteoconductive calcium phosphates are stiff, brittle and friable. As such they can easily fragment when loaded. In addition, the stress shielded environment within a porous but rigid material will inhibit new bone formation.

Natural scaffold materials such as collagen, which are derived from animal tissue, can elicit a foreign-body reaction and also the risk of disease transmission is always an issue of consideration. Collagen becomes very soft when wetted and as such does not provide any resistance to compressive forces once implanted. It will sag under its own weight when saturated with fluid.

A range of rapid prototyping techniques including selective laser sintering, fused deposition modelling, laminate object manufacture and inkjet printing have all been used to produce complex shaped 3D porous structures, in polymer and ceramic, for bodily implants. However these techniques can not achieve the level of fine detail, of the order of 100 microns, which is considered necessary for optimum cellular infiltration. In addition, their utility is generally limited to ‘custom’ implants, rather than mass-produced components.

According to the present invention there is provided a bioabsorbable material suitable for implanting within a human body, the material including fibres of a composite of a synthetic bioabsorbable polymer and a bioactive filler, the fibres being of non uniform cross section.

The fibres are preferably also of non uniform cross sectional area.

The fibres are preferably between 0.5 and 50 mm long.

The fibres preferably have a diameter range of between 3 and 300 microns.

The synthetic bioabsorbable polymer may be thermoplastic, and may comprise any of poly-L-lactic acid, poly DL-lactic acid, poly glycolide, poly caprolactone, poly dioxanone, poly hydroxybutyrate, poly hydroxyvalerate, poly propylene fumarate, poly ethylene-oxide, poly butylene terephthalate and mixtures, co-polymer or derivatives thereof.

The ratio of fibre length to diameter is preferably at least 10:1.

The bioactive filler may be osteoconductive, and may comprise alone or as mixtures hydroxyapatite, tri-calcium phosphate, calcium sulphate, calcium carbonate, bioactive glass or other bone inducing or cartilage inducing material.

The bioactive filler is preferably in the form of discrete particles distributed throughout the polymer fibres, and the filler preferably has a particle size of between 1 and 150 microns.

The fibres may be surface treated, and may be treated to impart hydrophilicity, surface electric charge, or surface coated to influence cell behaviour.

Preferably the material includes 5-80% by weight filler, and desirably 15-50% by weight filler.

The invention also provides a piece material, the material being formed from a bioabsorbable material according to any of the preceding ten paragraphs.

The piece material is preferably non woven, and may be in the form of a scaffold, fleece or felt.

The invention further provides a bone cement composition including a material according to any of the preceding twelve paragraphs as a reinforcement to the bone cement.

Embodiments of the present invention will now be described by way of example only and with reference to the accompanying drawings, in which:

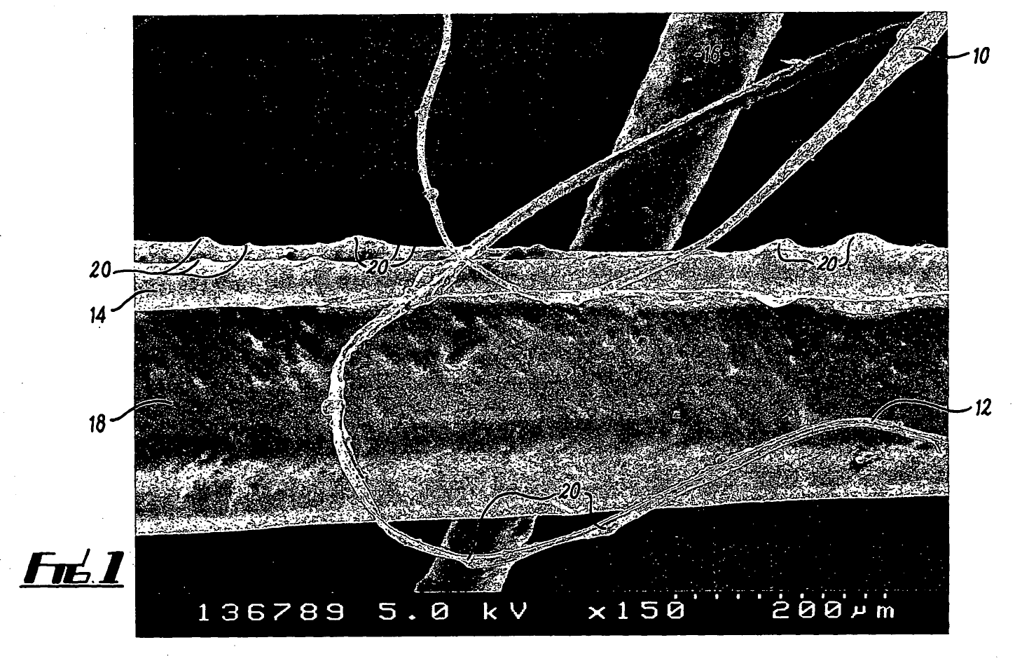

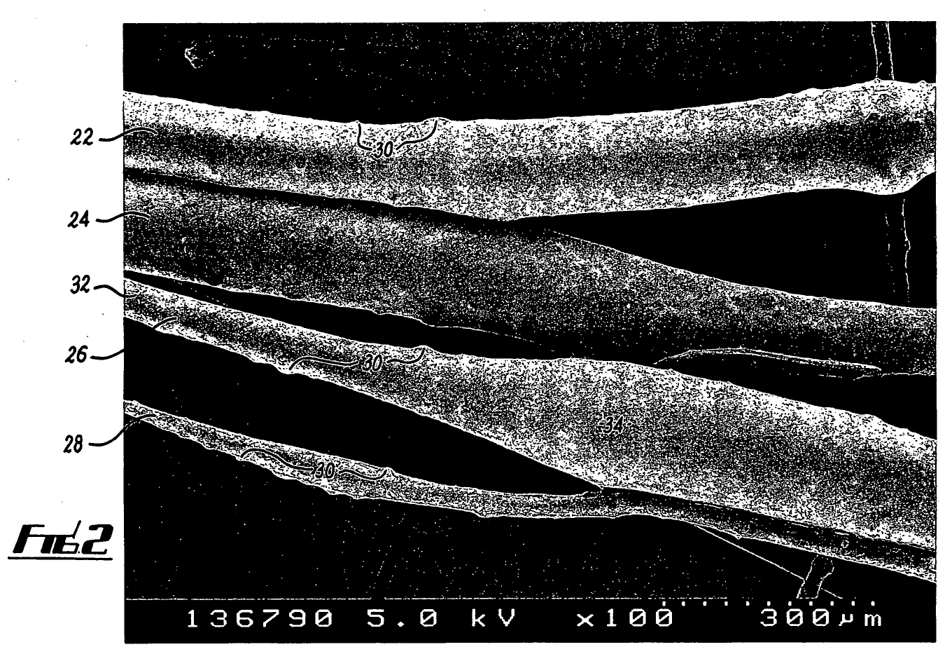

FIGS. 1 and 2 are scanning electron micrographs of fibres according to Example 1.

### Example 1

This is a fibrous bioabsorbable material as shown in FIGS. 1 and 2. The fibres consist of a synthetic bioabsorbable polymer such as poly-lactic acid and a particulate bioactive filler such as calcium phosphate powder. The fibres are discontinuous with lengths ranging from approximately one millimetre to several centimetres and diameters ranging from approximately 5 microns to approximately 300 microns. The diameter varies along the length of each fibre and the overall aspect ratio is at least 10:1 length: mean diameter. The filler particles which are distributed throughout the fibres are also evident as ‘bobbles’ on the surface of the fibres, and have a particle size range of approximately 1-150 microns.

FIG. 1 shows parts of five separate fibres 10, 12, 14, 16, 18. Fibre 10 has the smallest diameter of approximately 6 microns, whilst fibre 18 has the largest diameter of approximately 280 microns. The fibres 12, 14, 16 have intermediate diameters. The calcium phosphate powder particles present within the polymer are evident by various sized bobbles 20 on the surface of the fibres 10, 12, 14, 16, 18. The variation in diameter of the fibres 10, 12, 14, 16, 18 is apparent even within the restricted view of FIG. 1.

FIG. 2 shows four fibres 22, 24, 26, 28. The calcium phosphate particles present within the polymer are again shown by bobbles 30. the non uniform, irregular nature of the fibres 22, 24, 26, 28 along their length can clearly be seen. Fibre 26 is shown for example as varying from a diameter of approximately 50 microns at 32 to approximately 180 microns at 34, a distance of only approximately 700 microns.

### Example 2

A mixture of poly-lactic acid (PLA) and hydroxyapatite (HA) in the weight proportions 80:20 respectively was compounded into composite granules prior to melt spinning. The granule size was larger than the size of the orifice through which spinning was to take place while the particle size of the HA was less than the size of the orifice. The composite granules were fed into a cylindrical and axially rotatable holder, the outer circumferential surface of which consisted of a mesh or holed plate. A source of heat was provided to the holder to cause melting of the polymer component.

Rotation of the holder caused the composite granules to be forced centrifugally against the mesh or holed plate. The relative size difference between the holes and the granules prevented premature loss of granules through the holes. When heat was applied to the holder the polymer melted and the centrifugal force caused the pyroplastic composite to be forced through the holes to form fibres. As these fibres exited the holes outside the holder they cooled in the air stream and in so doing were stretched and broken into short lengths by the action of the rapidly rotating mesh or holed plate. The mesh size was 250 microns, the granule size 1-4 mm and the particle size of the HA 1-150 microns. The fibres had a diameter ranging from approximately 5 microns to approximately 200 microns and lengths from approximately 0.5 cm to approximately 5 cm.

The maximum diameter of the fibres is controlled by the diameter of the holes in the mesh or plate while the length of the fibres depends upon the particle size and quantity of the bioactive filler. Increasing the percentage fill of powder in the polymer and/or increasing the size of the powder particles will produce an overall reduction in the length of fibres produced.

### Example 3

Composite, tapered bioabsorbable fibres as described in example 1 and prepared as described in example 2 were surface treated to improve their hydrophilicity. This entailed soaking in an alkaline solution such as a saturated solution of lime water (calcium hydroxide) for a period of 4 hours at 37° C. The fibres were then washed free of solution, dried at 37° C., packaged in suitable containers and sterilised by gamma irradiation.

A small quantity of the sterile fibres, approximately one quarter of one cubic centimetre when compressed with light finger pressure, were packed into a freshly created tooth extraction socket where they immediately became saturated with blood. The blood clot which subsequently formed within the extraction socket, and which forms naturally following tooth extractions, held the fibres in place. Soft tissue formed over the clot as part of the normal healing process. Over a period of several months the polymer component resorbed and the osteoconductive nature of the calcium phosphate filler particles resulted in new bone formation within the socket. This subsequently helped to maintain ridge width and height.

Both radiographic and clinical evaluations of alveolar ridge dimensions following tooth extraction show significant loss of both width and height over time. This can make any subsequent treatments such as bridge or implant placements more difficult for the dentist or implantologist and less satisfactory for the patient. The fitting of dentures also becomes more problematic.

The technique described above can be performed simply and quickly by a general dental practitioner in the course of a normal tooth extraction procedure to help maintain ridge dimensions. This is a significant benefit to the patient as it can simplify subsequent treatments and improve treatment outcome both in terms of functionality and aesthetics.

### Example 4

A mixture of poly-L, DL (70/30) lactide and hydroxyapatite in the weight proportions 60:40 was processed into fibres as described in example 2. The HA had a particle size of 1-150 microns and the polymer had a molecular weight of 150,000 Daltons. The fibres had an aspect ratio of greater than 10 with a length range of approximately 0.5-4 millimetres. The diameters ranged from approximately 3 to 200 microns. These short fibres (whiskers) were used as a reinforcement in a calcium phosphate bone cement and as a bone graft containment mesh within a bony void, such as the cavity within a vertebral body.

There are thus described bioabsorbable fibres which provide for a number of advantages. Such fibres can be formed into non-woven materials such as scaffolds, felt or fleece. Such materials can easily be cut and compressed to fit the contours of a surgical defect to be filled. The stiffness of the scaffold can be controlled by the nature of the fibres, their composition and diameter, together with the level of entanglement and cross-bonding. The porosity is fully open and interconnected and the pore size easily controlled. The fibres can act as a continuous ‘pathway’ for the cells to invade the central depths of the scaffold.

The surface topography of the fibres together with the chemical nature of the bioactive filler particles provide a substrate that is more amenable to cellular colonisation than prior materials. The composite nature of the fibres increases their stiffness compared to polymer alone and hence gives a non-woven material which has an improved resistance to compression. Resistance to fibre pullout and fibre migration (in the absence of any cross bonding of the fibres) is improved by the tortuosity of the fibres, the rugosity of the fibre surface and the non-uniform diameter and cross-sectional area of the individual fibres.

Various modifications may be made without departing from the scope of the invention. The fibres can be used as formed, or can be used as a non-woven material. A single fibre type or a mixture of fibre types could be used to provide a specific functionality. The fibres may be processed into any physical form suitable for the intended application and may be used to support cell growth and tissue formation in vitro i.e. outside the body prior to implantation or in vivo i.e. implanted to a specific site to be seeded with cells in situ or allowed to be colonised by bodily cells in situ. The fibres or subsequent scaffold may be treated to impart hydrophilicity or surface electric charge, or surface coated to influence cell behaviour. The fibres or subsequent scaffold may be impregnated with bioactive molecules such as growth factors or morphogenic proteins. The scaffold may be functionally graded in terms of morphology and chemistry to provide features suitable for a combination tissue such as cartilage attached to sub chondral bone.

Such fibres could be mixed with material such as calcium phosphate or calcium sulphate powders and rehydrant solution to provide fibre reinforced bone graft cements having improved strength and toughness and a reduced potential to fragment.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1-20. (canceled)

21. A bioabsorbable material suitable for implanting within a human body, the material including fibers of a composite of a synthetic bioabsorbable polymer and a bioactive filler, the fibers being of non uniform cross section.

22. A material according to claim 1, wherein the fibers are also of non uniform cross sectional area.

23. A material according to claim 1, wherein the fibers are between 0.5 and 50 mm long.

24. A material according to claim 1, wherein the fibers have a diameter range of between 3 and 300 microns.

25. A material according to claim 1, wherein the synthetic bioabsorbable polymer is thermoplastic.

26. A material according to claim 1, wherein the synthetic bioabsorbable polymer comprises any of poly L-lactic acid, poly DL-Iactic acid, poly glycolide, poly caprolactone, poly dioxanone, poly hydroxybutyrate, poly hydroxyvalerate, poly propylene fumarate, poly ethylene-oxide, poly butylene terephthalate and mixtures, co-polymer or derivatives thereof.

27. A material according to claim 1, wherein the ratio of fibre length to diameter is at least 10:1.

28. A material according to claim 1, wherein the bioactive filler is osteoconductive.

29. A material according to claim 1, wherein the bioactive filler comprises alone or as mixtures hydroxyapatite, tri-calcium phosphate, calcium sulphate, calcium carbonate, bioactive glass or other bone inducing or cartilage inducing material.

30. A material according to claim 1, wherein the bioactive filler is in the form of discrete particles distributed throughout the polymer fibers.

31. A material according to claim 1, wherein the bioactive filler has a particle size of between 1 and 150 microns.

32. A material according to claim 1, wherein the fibers are surface treated.

33. A material according to claim 32, wherein the fibers are treated to impart hydrophilicity, surface electric charge, or surface coated to influence cell behavior.

34. A material according to claim 1, wherein the material includes 5-80% by weight filler.

35. A material according to claim 34, wherein the material includes 15-50% by weight filler.

36. A piece material, the material being formed from a bioabsorbable material according to claim 1.

37. A piece material according to claim 36, wherein the piece material is non woven.

38. A piece material according to claim 36, wherein the piece material is in the form of a scaffold, fleece or felt.

39. A bone cement composition including a material according to claim 21 as a reinforcement to the bone cement.
